(19) United States
(12) Patent Application Publication
(10) Pub. No.: US 2007/0287879 A1
(43) Pub. Date: Dec. 13, 2007

(54) Mechanical means for controlling blood pressure

(76) Inventors: Daniel Gelbart, (US); Samuel Victor Lichtenstein, (US)

Correspondence Address: SEED INTELLECTUAL PROPERTY LAW GROUP PLLC, 701 FIFTH AVE, SUITE 5400, SEATTLE, WA, 98104, US

(21) Appl. No.: 11/451,437

(22) Filed: Jun. 13, 2006

(51) Int. Cl. A61N 1/362

(52) U.S. Cl. 600/16

(57) ABSTRACT

Making the volume of the arterial system increase elastically with blood pressure reduces high systolic blood pressure peaks. This volumetric elasticity is achieved by the action of a spring controlling the aortic cross-section thus controlling the aortic volume. The spring can be implanted percutaneously. The device is powered by the blood pressure itself and requires no other energy source or control circuits. The device can have an open structure or a sealed-wall structure, the latter also serve to protect against aortic aneurism. Non-linear volumetric elasticity can be used to assist the heart.

### TECHNICAL FIELD

The disclosure relates to controlling high blood pressure by a simple implantable device and its potential for decreasing cardiac after load and increasing coronary perfusion.

### DESCRIPTION OF THE RELATED ART

High blood pressure is a very common disorder primarily caused by the major arteries losing flexibility over the years or smaller arterioles increasing vascular resistance. As the heart pumps the blood into the aorta, the aorta and other arteries behave as an elastic vessel expanding in order to absorb the newly injected volume of blood before it spreads in the body. This volumetric elasticity prevents the pressure from rising too high. With age and other factors arterioles increase their resistance and the large arteries loose their ability to expand in response to the pressure increase, resulting in high systolic pressure. The ability to elastically increase the volume in response to a pressure increase is referred to as “volumetric elasticity” in this disclosure.

Traditionally high blood pressure is treated by medication, with the well-known disadvantages of having to take regular medication, side effects, costs, need for continuous supply etc. For patients equipped with devices generally known as “heart-assist” devices or artificial hearts, these devices conceivably can be programmed to regulate the blood pressure and indeed such active means of controlling blood pressure and flow are well known in the literature. In this disclosure the term “active device” refers to a device that is powered by a power source, either internal or external to the body. Such devices usually also have electronic controls for regulating and monitoring their operation; some are fully programmable. As these devices require significant power, supplied by batteries or alternating current source, they have the major disadvantage of needing to keep these batteries charged either by surgical replacement or external means or limit the mobility of the patient. Another disadvantage is that all these devices require major surgery to be installed in the body.

A passive device (i.e., having no power source) is highly preferred because of simplicity, reliability, cost, and eliminating the need for a power source, which is generally the weak link in active devices.

Passive devices have been used to assist the heart but mainly in the form of components such as heart valves or external braces to strengthen and support the heart against the internal pressures. In general the purpose of the external wraps and braces applied to a weakened heart is to reduce the “volumetric elasticity” of the heart, as if the heart volume expands too easily with pressure it can not reduce its volume to expel the blood against the back pressure of the aorta. An example of an external device adding elasticity to the arterial system is disclosed in U.S. Pat. No. 4,938,766 however this device requires significant surgery in order to implant it inside the body. Internal devices are disclosed in US5409444 and US2004/0133260 however they significantly restrict the blood flow in the lumen, as they are based on adding an “hydraulic accumulator”.

### BRIEF SUMMARY

In one aspect, bringing back the required “volumetric elasticity” to the arteries, particularly the aorta, may limit pressure peaks and emulate a blood circulation system of a healthy person. Another aspect may limit and regulate blood pressure by using a flexure based passive device. Flexure based devices have no sliding parts that can wear out. Still another object is to make such a passive device small and simple, to enable implanting the device by minimally invasive surgery or by using a catheter. In still another aspect a device, which can by implanted inside the aorta or other large arteries may protect against aneurysm formation. Further advantages will become apparent by reading the disclosure in conjunction with the drawings.

In at least one embodiment, making the volume of the arterial system increase elastically with blood pressure reduces high systolic blood pressure peaks. This volumetric elasticity is achieved by the action of a spring controlling the aortic cross-section thus controlling the aortic volume. The spring can be implanted percutaneously. The device is powered by the blood pressure itself and requires no other energy source or control circuits. The device can have an open structure or a sealed-wall structure, the latter also serve to protect against aortic aneurysm. Non-linear volumetric elasticity can be used to assist the heart.

### BRIEF DESCRIPTION OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the Darticular shaDes of the elements as drawn, are not intended to convey any information regarding the actual shape of the particular elements, and have been solely selected for ease of recognition in the drawings.

FIG. 1 is a partial isometric view of a device installed on the aorta according to one embodiment, the aortic wall cut away for clarity.

FIG. 2-a is a cross sectional view of the device of FIG. 1 at a moment of diastolic pressure.

FIG. 2-b is a cross sectional view of the device of FIG. 1 at a moment of peak systolic pressure.

FIG. 3 is a partial isometric view of the device of FIG. 1 being installed inside the aorta via a catheter.

FIG. 4 is a partial isometric view of the device according to an alternate embodiment inserted into an aorta, the device also capable of controlling aortic aneurysm.

FIG. 5-a is a cross sectional view of the internal elastic device of FIG. 4 at the moment of diastolic pressure.

FIG. 5-b is a cross sectional view of the device of FIG. 4 at a moment of peak systolic pressure.

FIG. 6 is a partial isometric view of the device of FIG. 4 illustrated in a folded position and being delivered via a catheter.

FIG. 7 is a graph showing an the effect of the device on systolic blood pressure according to one exemplary embodiment.

FIGS. 8a-8c are isometric views of a device experiencing three different pressures in an aorta, the device having a highly non-linear relationship between volume and pressure, useful in assisting the heart.

FIG. 9 is a front plan view of the device according to another alternate embodiment, which allows a smaller diameter catheter to be used for delivery.

FIG. 10 is a front plan view of the device according to still another embodiment which eliminated the need for a polymeric coating.

### DETAILED DESCRIPTION

In some embodiments restores the lost volumetric elasticity to the arteries by first decreasing the volume inside the arteries, allowing it to increase elastically as the pressure goes up. Since the amount of blood pumped out by the heart into the aorta during each contraction is about 60 cubic centimeter (cc), even a change in volume as small as 5_cc during each heartbeat will have an effect on systolic blood pressure and increasing the aortic volume by 10-20_cc will reduce an abnormally high blood pressure to a normal value. Since the change in volume of the blood in the aorta is comparable to the volume pumped out with each contraction, changing the volume of the aorta by 10 to 20% is sufficient to prevent high blood pressure and can be accomplished by an internal elastic device. An internal elastic device can be implanted percutaneously and eliminates the need for surgically opening the chest cavity. An internal elastic device can be inserted into the aorta through a major artery such as in the leg, similar to balloon insertion done today for angioplasty. The materials used, and design details, are critical for two main reasons:
1. The device flexes each time the heart beats thus the lifetime should be in the order of a few billion flexing cycles without a failure.
2. Materials should be compatible with blood and body tissue.

The fatigue life of an elastic element made of metal can be made practically infinite if proper design is used. For example, the hairspring (the escapement spring) in a mechanical watch beats about five times faster than the human heart and lasts a lifetime. This is possible because of phenomena known as “endurance limit” in highly elastic metals such as heat-treated steels. This means that if a spring is stressed below a certain stress level (about 50% of the ultimate tensile strength for hardened steel) fatigue life will be billions of cycles and failures will be random. In order to further reduce chances of random fatigue failure in the preferred embodiment the stress levels in the material are kept below 30% of the ultimate tensile stress and the stressed areas are free of scratches, as defects and scratches can start a fatigue failure. The design theory for the elastic elements is well known to mechanical engineers and is also available online (including software for design optimization), for example at:

<http://www.engrasp.com/products/etbx/library/fm/index.jsp>

When also considering the second requirement of compatibility with the human body, the best materials for the spring are spring tempered (hard) stainless steels, series 300, 400 or heat-treated 17-7 steel, plated heat-treated beryllium copper and Nitinol. There are many other materials compatible with the human body but most have a lower endurance limit. This subject is also well known in the medical art as many implants are used today. As far as polymeric coating materials, silicone rubber, Teflon, Dacron and others can be used. All these materials are well known in the art of medical devices. As is common practice with such devices, the device can be coated with drug-eluting coatings and other functional coatings well known in the art of stents.

Various embodiments take advantage of the fact that for a given perimeter length, a circle has the largest cross section (i.e., area). As the circle is deformed into an oval shape the area is reduced, all the way to zero when the circle is flattened into a line. Since the aortic volume is simply the cross sectional area times the length, very large changes in aortic blood volume can be achieved by changing the cross section. If the change is done by an elastic device, the lost volumetric elasticity of the arterial system can be restored and blood pressure lowered. By the way of example, assuming an aortic diameter of 3_cm, the cross section when round is about 7 sq. cm. When flattened to An oval about 1×4_cm without changing the perimeter length, the area is about 4_sq.cm. For a 20_cm long section of the aorta this represents a volumetric change of 20(7-4)=60_cc, which is nearly as large as the whole cardiac stroke volume. Such a change can easily reduce systolic blood pressure from 180_mm Hg to 120_mmHg.

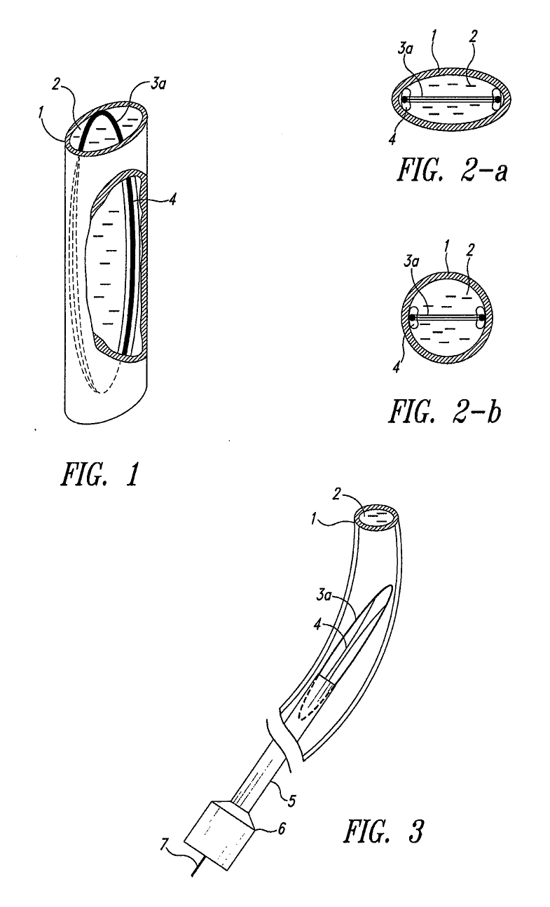

Referring now to FIG. 1, an aorta 1 is filled with blood 2 and contains an elastic oval ring or spring 3a. The elongated sides of the ring or spring 3a are coated with a soft polymeric coating 4 in order to distribute the load on the aortic walls. In a relaxed state, the elastic oval ring or spring 3a is significantly wider than the aorta, so it forms a low k high x spring when installed (the terms k and x are spring constant and initial displacement (also known as “preload”) from the spring formula: force=k.x). The elastic oval ring or spring 3a deforms the aorta into an oval cross section (low blood volume) as shown in FIG. 2-a . The systolic pressure overcomes the force of the elastic oval ring or spring 3a and restores the aorta to a more rounded position, as shown in FIG. 2-b. In order to insert the elastic oval ring or spring 3a via a catheter the elastic oval ring or spring 3a is fully compressed as shown in FIG. 3. A catheter 5 is similar to those used in other percutaneous cardiac procedures and typically has a foam seal 6 to avoid blood loss and a push wire 7 to deploy the device. By the way of example, the elastic oval ring or spring 3a is stainless steel spring wire having a diameter of 0.8-1_mm, the length of elastic oval ring or spring 3a is about 25_cm. The spring 3acan be inserted via an 8_mm ID catheter or even smaller when ribbon is used instead of wire. The polymeric coating 4 is 8_mm wide by 2_mm thick silicone rubber. In a simulated human artery the spring 3a reduced peak systolic pressure from 180 to 120_mmHg.

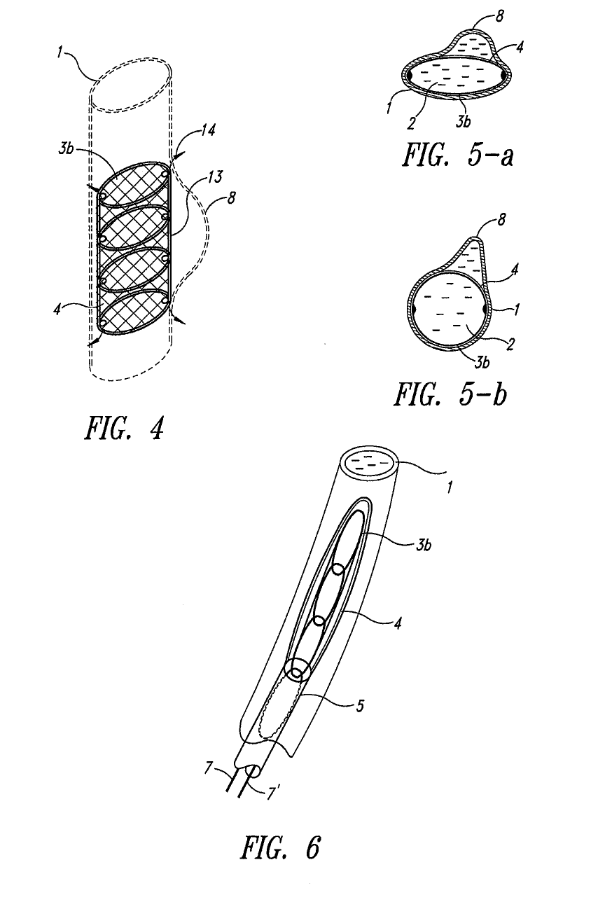

An alternate embodiment is shown in FIG. 4. A set of elastic ovals rings or springs 3b are linked by elastic links 13 and wrapped in a continuous polymeric coating 4. Since the coating 4 is a continuous sleeve, the coating 4 can also seal defects in the artery such as an aneurysm 8. FIG. 5-a shows the diastolic shape of the elastic oval rings or springs 3b and elastic links 13 and FIG. 5-b is the systolic shape of the elastic oval rings or springs 3b at the point of peak pressure. The elastic oval rings or springs 3b and elastic links 13 are flexible and can be folded as shown in FIG. 6 in order to fit into the catheter 5. Push wire 7 can be augmented by pull wire 7′ to assist in unfolding the elastic oval rings or springs 3b. This embodiment requires a high degree of elasticity and the preferred embodiment is made of heat treated Nitinol wire, typically 0.4-0.8_mm diameter or Nitinol ribbon. The elastic links 13 can simply be bent around the elastic oval rings or springs 3b for ease of folding. In such a case, location barbs 14 are desirable to anchor the unfolded structure to the aorta wall. Barbs 14 can simply be an extension of the elastic links 13.

The effect of the devices on systolic pressure is shown in FIG. 7. Graph 9 shows systolic pressure as a function of blood volume ejected from the left ventricle. When aorta is inelastic, blood pressure rises rapidly with volume. When the elastic device is installed, the graph follows curve 10. Depending on the exact spring constant k and preload x chosen, the shape of curve 10 can be customized. A lower spring constant k yields a flatter curve. For a given pressure change, a lower spring constant k requires a larger preload, as the total force should be the same. The spring constant k is calculated based on the well known formula: P.deltaV=0.5 k(systolic x̂2-diastolic x̂2). P is the blood pressure, delta V is the arterial volume change. P times delta V is simply the change of energy, which equals the change in energy stored in the spring. The value of the spring constant k should be corrected for the natural elasticity of the aorta and surrounding tissue, thus the spring constant k is larger than the value given by the formula. The preload is calculated based on the point where the volume should start changing: a large preload means no volume change till a certain pressure. A low spring constant k and large preload system behaves like graph 14 in FIG. 7, while a higher spring constant k and lower preload behaves like graph 10.

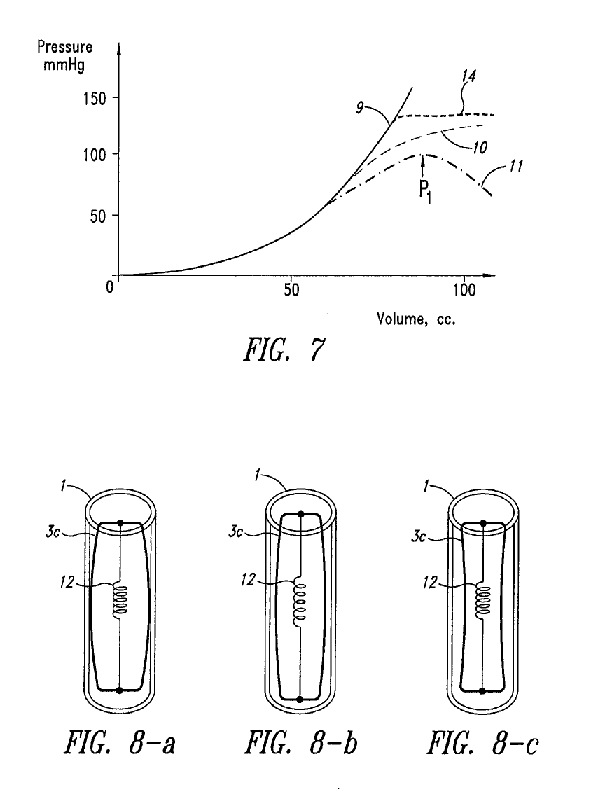

A customization for a heart condition of particular interest is the use of non-linear volume change to decrease after load and increase diastolic coronary perfusion in a compromised heart—not unlike an intra-aortic balloon pump. If in FIG. 7 graph 11, at given pressure point P1 the change in aortic blood volume capacity was not slow but abrupt, this could decrease cardiac after load and increase coronary perfusion independently of blood pressure control. For example, if repeatedly at 100 mmHg blood pressure during cardiac systole aortic blood volume capacity is suddenly increased, the resultant sudden decrease in aortic pressure would help the heart to better empty itself into a low pressure system. The result would be an increased stroke volume and cardiac output. If repeatedly at a blood pressure of, say, 80 mmHg, during cardiac diastole, there was a sudden decrease in aortic blood volume capacity diastolic blood pressure would increase and augment coronary and renal perfusion. This can be achieved by using an elastic member with non-linear elastic properties and in particular a spring with a negative spring constant k over part of the travel, better known as “snap action”. Such a spring system is shown in FIG. 8. As the pressure in the aorta 1 increases, a firstspring 3c flattens and elongates as shown in FIG. 8a and 8b. Additional a second spring 12 elongates as the first elastic oval ring or spring 3c narrows. Any increased pressure beyond FIG. 8b (corresponding to point P1 in FIG. 7 graph 11) will cause the first elastic oval ring or spring 3c to snap to position shown as FIG. 8c. Such a snap increases the volume of the aorta suddenly and assist the heart, as it actually pulls blood from the heart. The same beneficial effect is achieved during the diastole. This arrangement better matches the output of a volume loaded weak heart to the fluidic impedance of the arterial system. It can be tailored with great flexibility, as there are at least four parameters to adjust independently: spring constant k, preload, snap point and amount of snap.

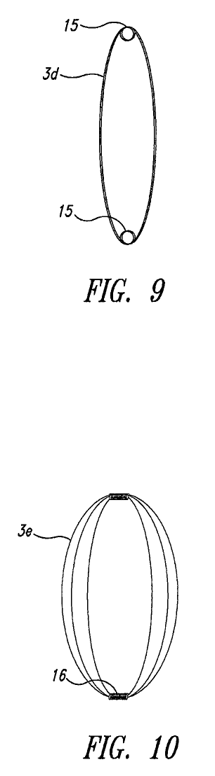

To further reduce the catheter size needed for implanting the device the configuration shown in FIG. 9 can be used. An elastic member 3d has a small coil 15 at both ends. Such a coil greatly increases elasticity, allowing compressing the device into a very small catheter. By the way of example, using 1_mm spring wire and a 3_mm diameter coils the device fits into a 4_mm ID catheter. Even a thinner wire and smaller catheter can be used when a single elastic member 3d is replaced by a chain made of multiple elastic members 3d, each plastic member 3d resembling that shown in FIG. 9. Another advantage of a chain-like device is greater ability to conform to the aortic longitudinal shape.

FIG. 10 shows an elastic device made from multiple thin wires instead of single wire or ribbon. The advantage of this configuration is that the load can be spread on the aortic wall without use of a polymeric coating. Wires 3e are twisted together at each end 16 in order to reduce the obstruction to flow of blood. In one aspect. a method for controlling blood pressure comprises adding volumetric elasticity to the blood circulation system by implanting a passive device inside the blood circulation system, said device having no enclosed volume. The device can be implanted percutaneously. The relationship between blood pressure and volume increase may be non-linear. The passive device may reduce aortic cross section by less than 10% during systolic pressure.

In another aspect. a method for controlling blood pressure comprises implanting an elastic member inside the aorta, said member adding volumetric elasticity to the aorta by making the cross section of the aorta change with blood pressure. The member may be made of flexible wire and can be implanted percutaneously. The member may be covered by a hemostatic coating in order to seal off parts of the aorta wall. The member may also be used to assist the heart. The member may be attached to the wall of the aorta by barbs. The member may be made of flexible wire in the shape of an elonciated oval, and said oval is partially covered by a non-metallic coating. The member may be coated with a drug eluting coating.

In yet another aspect. a device for controlling blood pressure allows the aorta to elastically increase its volume as blood pressure increase, said device reducing the volume of the blood in the aorta at low blood pressure by deforming the cross section of the aorta from circular to an elongated oval.

In the above description. certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with implantable devices have not been shown or described in detail to avoid unnecessarily obscuring descriptions of the embodiments.

Unless the context requires otherwise. throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Further more, the particuIar features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not internret the scope or meaning of the embodiments.

The above description of illustrated embodiments, including what is described in the Abstract, is not intended to be exhaustive or to limit the embodiments to the precise forms disclosed. Although specific embodiments of and examples are described herein for illustrative purposes, various equivalent modifications can be made without delparting from the spirit and scope of the disclosure, as will be recognized by those skilled in the relevant art.

These and other changes can be made to the embodiments in light of the above-detailed description. In general. in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-12. (canceled)

13. A passive device to control blood pressure, comprising:
at least a first member sized to be received within an inside of a human aorta and to physically engage the inside of the human aorta such that the first member will physically deform a first portion of the human aorta to have a smaller cross sectional area when the first portion of the human aorta is subjected to a diastolic pressure than the first portion of the human aorta would otherwise have if not deformed by the first member.

14. The passive device of claim 13 wherein the first member has a spring constant, the spring constant such that the first cross sectional area of the first portion of the human aorta is smaller than a cross sectional area of the first portion of the human aorta when the first portion of the human aorta is subjected to a systolic pressure.

15. The passive device of claim 14 wherein the first member comprises a material selected from the group consisting of spring tempered stainless steel, series 300 steel, series 400 steel, heat-treated 17-7 steel, plated heat-treated beryllium copper and Nitinol.

16. The passive device of claim 14 wherein the first member includes a first elastic member that has a spring constant, the spring constant such that the first cross sectional area of the first portion of the human aorta is smaller than a cross sectional area of the first portion of the human aorta when the first portion of the human aorta is subjected to a systolic pressure.

17. The passive device of claim 16 wherein the first member is a first elastic ring elastically deformable between a relaxed shape enclosing a relaxed area and an unrelaxed shape enclosing an unrelaxed area, the unrelaxed area less than the relaxed area.

18. The passive device of claim 17 wherein the relaxed shape is more circular than the unrelaxed shape.

19. The passive device of claim 17 wherein the relaxed shape is circular and the unrelaxed shape is oval.

20. The passive device of claim 17, further comprising:
a second elastic ring elastically deformable between a relaxed shape and an unrelaxed shape, the second elastic ring sized to be received within the inside of the human aorta and to physically engage the inside of the human aorta such that the second elastic ring will physically deform a second portion of the human aorta to have a first cross sectional area when the second portion of the human aorta is subjected to the diastolic pressure than the second portion of the human aorta would otherwise have if not deformed by the second elastic ring; and
a first number of elastic link members linking the first and the second elastic rings.

21. The passive device of claim 20, further comprising:
at least one barb extending outwardly from the passive device.

22. The passive device of claim 20, further comprising:
a third elastic ring elastically deformable between a relaxed shape and an unrelaxed shape, the third elastic ring sized to be received within the inside of the human aorta and to physically engage the inside of the human aorta such that the third elastic ring will physically deform a third portion of the human aorta to have a third cross sectional area when the third portion of the human aorta is subjected to the diastolic pressure that is smaller than the third portion of the human aorta would otherwise have if not deformed by the third elastic ring; and
a second number of elastic link members linking the second and the third elastic rings.

23. The passive device of claim 22, further comprising:
a fourth elastic ring elastically deformable between a relaxed shape and an unrelaxed shape, the fourth elastic ring sized to be received within the inside of the human aorta and to physically engage the inside of the human aorta such that the fourth elastic ring will physically deform a fourth portion of the human aorta to have a first cross sectional area when the fourth portion of the human aorta is subjected to the diastolic pressure that is smaller than a cross sectional area that the fourth portion of the human aorta would otherwise have if not deformed by the fourth elastic ring; and
a third number of elastic link members linking the third and the fourth elastic rings.

24. The passive device of claim 17 wherein the first elastic ring includes a first coil and a second coil, the second coil opposed across the first elastic ring from the first coil.

25. The passive device of claim 17, further comprising:
a plurality of additional elastic rings commonly coupled together at respective portions thereof with the first elastic ring.

26. The passive device of claim 13, further comprising:
a connecting spring that connects at least two portions of the first member and biasing the first member to snap into a shape that would increase a volume of the human aorta.

27. The passive device of claim 13 wherein the passive device fits inside a 4 millimeter inner diameter catheter when compressed.

28. The passive device of claim 13 wherein the passive device decreases a volume of the human aorta by between 10 percent and 20 percent when the first portion of the human aorta is subjected to the diastolic pressure.

29. The passive device of claim 13 wherein the passive device decreases a volume of the human aorta by about 60 cubic centiliters.

30. The passive device of claim 13 wherein the passive device is an open wall structure.

31. The passive device of claim 13 wherein first member is sized to physically deform the first portion of the human aorta to have a first cross sectional area when the first portion of the human aorta is subjected to the diastolic pressure, the first cross sectional area being smaller than a cross sectional area that the first portion of the human aorta would have if not deformed by the first member subjected to the diastolic pressure.

32. A method of forming a passive device to control blood pressure, the method comprising:
providing a first member; and
forming the first member into a size and shape to physically engage an inside of the human aorta such that a first portion of the human aorta will be physically deformed to have a relatively smaller cross sectional area when the first portion of the human aorta is subjected to a diastolic pressure than the human aorta would otherwise have.

33. The method of claim 32 wherein providing a first member includes providing one of at least first one of a wire or a ribbon of a spring material.

34. The method of claim 33 wherein forming the first member into a size and shape to physically engage an inside of the human aorta includes forming a first elastic ring from the at least first one of the wire or the ribbon of a spring material.

35. The method of claim 34, further comprising:
providing a second one of at least one of a wire or a ribbon of a spring material; and
forming the second one of at least one of the wire or the ribbon into a second elastic ring sized and shaped to physically engage the inside of the human aorta such that a second portion of the human aorta will be physically deformed to have a relatively smaller cross sectional area when the second portion of the human aorta is subjected to a diastolic pressure than the human aorta would otherwise have; and
connecting the first and the second elastic rings with a number of elastic links.

36. The method of claim 34 wherein forming the first elastic ring includes forming a first loop and a second loop in the first elastic ring, the first and the second loops opposed to one another.

37. The method of claim 34, further comprising:
connecting portions of the first elastic ring with a connecting spring to provide a nonlinear spring constant thereto.

38. The method of claim 34, further comprising:
providing a plurality of additional ones of at least one of a wire or a ribbon of a spring material; and
forming each of the additional ones of at least one of the wire or the ribbon into a plurality of additional elastic rings sized and shaped to physically engage the inside of the human aorta such that a the human aorta will be physically deformed to have a relatively smaller cross sectional area when the human aorta is subjected to a diastolic pressure than the human aorta would otherwise have; and
commonly connecting potions of the first and the additional elastic rings.

39. A passive device to control blood pressure, comprising:
means for physically engaging an inside of the human artery such that a first portion of the human artery will be physically deformed to have a relatively smaller cross sectional area when the first portion of the human artery is subjected to a diastolic pressure than the human artery would otherwise have.

40. The passive device of claim 39 wherein the means comprises a first elastic ring elastically deformable between a relaxed shape and an unrelaxed shape, the first elastic ring sized to be delivered percutaneously.

41. The passive device of claim 40 wherein the first elastic ring is formed from at least one of an elongated wire or an elongated ribbon.

42. The passive device of claim 39 wherein the means comprises a material selected from the group consisting of spring tempered stainless steel, series 300 steel, series 400 steel, heat-treated 17-7 steel, plated heat-treated beryllium copper and Nitinol.

43. The passive device of claim 39 wherein the means comprises a plurality of elastic rings elastically deformable between a relaxed shape and an unrelaxed shape, and a plurality elastic links linking respective pairs of the elastic rings, the elastic rings and the elastic links deformable to be delivered percutaneously as a unit.

44. The passive device of claim 43 wherein at least one of the elastic links forms at least one barb extending outwardly from the passive device.

45. The passive device of claim 39 wherein the means comprises a first elastic ring elastically deformable between a relaxed and an unrelaxed shape, and a connecting spring connecting at least two portions of the first elastic ring to bias the first elastic ring to snap into a shape that would increase a volume of the human artery.

46. The passive device of claim 39 wherein the means includes a first elastic ring that is elastically deformable, the first elastic ring having a first coil therein and a second coil therein, the second coil opposed across the first elastic ring from the first coil.

47. The passive device of claim 39 wherein the means includes a plurality of elastic rings commonly coupled together at respective ends thereof, each of the elastic rings elastically deformable between a relaxed shape and an unrelaxed shape, the elastic rings each sized to be percutaneously delivered within the human.

48. The passive device of claim 39 wherein the human artery is an aorta.

49. A method employing a passive device, comprising:
positioning a catheter bearing a first member in an inside of a human artery; and
implanting the first member sized to be received within the inside of the human artery and to physically engage the inside of the human artery such that the first member will physically deform a first portion of the human artery to have a first cross sectional area when the first portion of the human artery is subjected to a diastolic pressure, that is smaller than the first portion of the human artery would otherwise have.

50. The method of claim 49 wherein implanting a first member includes implanting a first member having a spring constant, the spring constant such that the first cross sectional area of the first portion of the human artery is smaller than a cross sectional area of the first portion of the human artery when the first portion of the human artery is subjected to a systolic pressure.

51. The method of claim 49 wherein implanting a first member includes implanting the first member includes implanting a first elastic member that has a spring constant, the spring constant such that the first cross sectional area of the first portion of the human artery is smaller than a cross sectional area of the first portion of the human artery when the first portion of the human artery is subjected to a systolic pressure.

52. The method of claim 51 wherein implanting a first member includes implanting a first elastic ring elastically deformable between a relaxed shape enclosing a relaxed area and an unrelaxed shape enclosing an unrelaxed area, the unrelaxed area less than the relaxed area.

53. The method of claim 52 wherein the relaxed shape is more circular than the unrelaxed shape.

54. The method of claim 49 wherein implanting a first member includes implanting a first member comprising a material selected from the group consisting of spring tempered stainless steel, series 300 steel, series 400 steel, heat-treated 17-7 steel, plated heat-treated beryllium copper and Nitinol.

55. The method of claim 49 wherein the human artery is an aorta and implanting the first member includes implanitng the first member to physically engage the inside of the aorta.
